(19) United States
(12) Patent Application Publication
(10) Pub. No.: US 2023/0390514 A1
(43) Pub. Date: Dec. 7, 2023

(54) Systems and Methods for Reforming Wearable Devices

(71) Applicants: Alexander Simes, Saint Johns, FL (US); Emma Ostrout, Saint Johns, FL (US); Jaiden Torres, Saint Johns, FL (US); Jacob Denson, Saint Johns, FL (US); Dennis Dohogne, Saint Johns, FL (US); Kevin Davenport, Saint Johns, FL (US)

(72) Inventors: Alexander Simes, Saint Johns, FL (US); Emma Ostrout, Saint Johns, FL (US); Jaiden Torres, Saint Johns, FL (US); Jacob Denson, Saint Johns, FL (US); Dennis Dohogne, Saint Johns, FL (US); Kevin Davenport, Saint Johns, FL (US)

(21) Appl. No.: 18/206,586

(22) Filed: Jun. 6, 2023

Related U.S. Application Data
(60) Provisional application No. 63/349,612, filed on Jun. 7, 2022.

(51) Int. Cl. A61M 16/06

(52) U.S. Cl. CPC A61M 16/0605; A61M 2016/0661; A61M 2202/0208; A61M 2207/10

(57) ABSTRACT

The present disclosure provides for systems and methods for reforming wearable devices. The system may comprise a wearable device. The wearable device may comprise a gasket. The wearable device may comprise a pliable region. The pliable region may comprise a predefined diameter to interface with a plurality of wearable devices. The pliable region may comprise the gasket. The wearable device may comprise a supply device. The wearable device may comprise an attachment mechanism. The wearable device may interface with a reforming device. The reforming device may interface with a scanner. The reforming device may adjust the wearable device based on information received from the scanner. The reforming device may receive information from an external device. The external device may comprise a scanner that sends information to the reforming device.

### CROSS-REFERENCE TO RELATED APPLICATION

This application claims priority to and the full benefit of U.S. Provisional Patent Application Ser. No. 63/349,612, filed Jun. 7, 2022, and titled “SYSTEMS AND METHODS FOR REFORMING WEARABLE DEVICES”, the entire contents of which are incorporated in this application by reference.

### BACKGROUND

With the introduction of prolonged medical treatment and long-term implants, the challenge of comfort and personalized fit has continued to challenge both provider and patient alike. The provider has struggled to utilize a device that can be adaptable to each patient to reduce cost through reusability. The patient has struggled to find treatment solutions that are comfortable to wear or use over a prolonged period of time.

When a medical device is used consistently over a length of time, comfort and a personalized fit become more critical to avoid improper fit complications such as chafing, blistering, and subsequent infection. Sometimes persistent complications even result in scarring. This challenge is exemplified in the endeavors undertaken to make oxygen and other long-term masks more comfortable.

For example, people with sleep apnea often struggle to find a mask that fits well enough for sustained use. Because sleep apnea often causes abrupt stops in breathing while sleeping, it is imperative that these people can wear a mask that is comfortable enough to sleep in and has a secure fit to supplement oxygen flow when necessary.

Face masks can deliver necessary medical gasses such as oxygen or anesthesia, such as in a hospital application. In other situations, face masks can exclude harmful gasses and liquids from the user's nose and mouth, such as a firefighter's mask. The face masks are often constructed in shapes that provide a general fit to a human face. In order to achieve a suitable seal, the masks are typically made of semi-pliable or semi-conformable materials, especially at the interface to the skin. Additionally, straps typically secure the mask in place and apply forces to form a seal between the mask and skin. The semi-pliable materials and construction have enough rigidity for requisite attachment to other parts of the mask, such as hose connections and strap attachments, and retain their shape. These masks are made for a general-purpose fit and rely on strong and uncomfortable strap forces to achieve a suitable seal.

Trying to find a way to make masks more comfortable has been a goal that dates as far back as 1998, when there was an attempt to make a face mask from a facial impression. However, this solution continues to remain costly and time consuming, as the multi-step process requires new material to cast a new face mask whenever the fit becomes improper. Alternatively, the straps attached to the mask have become adjustable, but improper fits remain an expensive correction.

Others have tried to solve the problem with one-time fixes such as facial impressions, using plastic polymers, or other moldable materials, creating custom molds, and providing a plurality of available mask sizes. However, a common problem with the universal size persists by providing a predefined shape with an imperfect fit. The only remedy comprises the purchase and casting of a new mask, which continues to be a costly and time-consuming process.

### SUMMARY OF THE DISCLOSURE

What is needed is a wearable device that may be reshaped in accordance with the changing dimensions of a user's face. The wearable device may allow the user to reshape the wearable device in accordance with the shape of the user's face shape. The wearable device may provide a customized wearable device, such as a face mask for Continuous Positive Airway Pressure (CPAP) users, by way of example and not limitation. Using the CPAP masks as a continuing example, secure sealing and comfort are the biggest problems with these devices. The wearable device may provide an easy means for the mask wearer to custom fit the mask themselves, with the ability to reshape the fit of the mask when necessary.

The wearable device addresses patient needs by providing a means to custom-fit the wearable device to the individual user for a more secure seal and a more comfortable fit than non-customizable alternatives. Further, the wearable device is easily reformed to allow the user to change the fit at their discretion. This is valuable because the patient's facial features might change due to weight gain or loss, or even due to changes in hair styles or facial hair. Also, a patient might decide to refit the wearable device to achieve a better fit than their current fit or to refit the wearable device due to material deformation from prolonged use.

In some embodiments, the wearable device may comprise a modified sleep apnea product. The wearable device may utilize a chemical compound that softens when heated and conforms to facial features. In some implementations, the wearable device may utilize a reforming process comprising an ultrasonic treatment, photonic treatment, electrical treatment such as nitinol or shape-memory wires, air-pressurized compressive systems, or a ferrofluidic treatment, as a list of non-limiting examples.

Reshaping the wearable device may alleviate pressure on the user by providing more comfort and improved lifespan of the mask. The wearable device may allow for repeated reshaping so a user may continue to use the wearable device when the user undergoes changes such as weight loss or gain. In some implementations, the wearable device may allow the user to reshape the wearable device without having to go through the process of repurchasing a new wearable device.

The present disclosure provides for systems and methods for reforming wearable devices. The system may comprise a wearable device. The wearable device may comprise a gasket. The wearable device may comprise a pliable region. The pliable region may comprise a predefined diameter to interface with a plurality of wearable devices. The pliable region may comprise the gasket. The wearable device may comprise a supply device. The wearable device may comprise an attachment mechanism.

The wearable device may interface with a reforming device. The reforming device may interface with a scanner. The reforming device may adjust the wearable device based on information received from the scanner. The reforming device may receive information from an external device. The external device may comprise a scanner that sends information to the reforming device.

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1A illustrates an exemplary wearable device, according to some embodiments of the present disclosure.

FIG. 1B illustrates an exemplary wearable device, according to some embodiments of the present disclosure.

FIG. 1C illustrates an exemplary wearable device, according to some embodiments of the present disclosure.

FIG. 2A illustrates an exemplary wearable device, according to some embodiments of the present disclosure.

FIG. 2B illustrates an exemplary wearable device, according to some embodiments of the present disclosure.

FIG. 3A illustrates an exemplary wearable device, according to some embodiments of the present disclosure.

FIG. 3B illustrates an exemplary wearable device, according to some embodiments of the present disclosure.

FIG. 4 illustrates an exemplary wearable device interfacing with a reforming device, according to some embodiments of the present disclosure.

FIG. 5 illustrates an exemplary wearable device interfacing with a reforming device, according to some embodiments of the present disclosure.

FIG. 6A illustrates an exemplary wearable device interfacing with a reforming device, according to some embodiments of the present disclosure.

FIG. 6B illustrates an exemplary wearable device interfacing with a reforming device, according to some embodiments of the present disclosure.

FIG. 7 illustrates an exemplary wearable device interfacing with a reforming device, according to some embodiments of the present disclosure.

FIG. 8 illustrates an exemplary wearable device interfacing with a reforming device, according to some embodiments of the present disclosure.

FIG. 9 illustrates an exemplary wearable device interfacing with a reforming device, according to some embodiments of the present disclosure.

FIG. 10A illustrates an exemplary wearable device interfacing with a reforming device, according to some embodiments of the present disclosure.

FIG. 10B illustrates an exemplary wearable device, according to some embodiments of the present disclosure.

FIG. 11 illustrates an exemplary wearable device interfacing with a reforming device, according to some embodiments of the present disclosure.

FIG. 12 illustrates an exemplary wearable device, according to some embodiments of the present disclosure.

FIG. 13 illustrates exemplary method steps for reforming wearable devices, according to some embodiments of the present disclosure.

### DETAILED DESCRIPTION

The present disclosure provides generally for systems and methods for reforming wearable devices. The system may comprise a wearable device. The wearable device may comprise a gasket. The wearable device may comprise a pliable region. The pliable region may comprise a predefined diameter to interface with a plurality of wearable devices. The pliable region may comprise the gasket. The wearable device may comprise a supply device. The wearable device may comprise an attachment mechanism.

In the following sections, detailed descriptions of examples and methods of the disclosure will be given. The descriptions of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Wearable device: as used herein, refers to any device that may be worn by a user that comprises a fitting designed to form a secure seal with at least one portion of an interior or exterior surface of the user's body, such as, for example and not limitation, one or more portions of the user's skin. In some embodiments, the wearable device may comprise at least one supply device that may interface with at least one external device, such as an oxygen supply system. In some aspects, a user may comprise a human, animal, or inanimate object, as non-limiting examples. By way of example and not limitation, a user surface may comprise one or more portions of a face, mouth, arm, tooth, leg, or any other internal or external surface of a user, as non-limiting examples.
Scanner: as used herein, refers to a device that records dimensions of a surface that may comprise a plurality of contours. In some embodiments, the scanner may comprise a measurement device that may measure dimensions via optical devices, infrared devices, as non-limiting examples. In some implementations, the scanner may generate digital information that describes facial contours and topology. In some aspects, the scanner may interface with external devices such as a camera and third-party software, as non-limiting examples.
Gasket: as used herein, refers to a portion of a wearable device that provides a customized fit to at least a portion of a user's body, such as, for example and not limitation, one or more portions of a user's face. In some embodiments, the gasket may provide a seal. In some implementations, a pliable region may comprise a gasket. In some aspects, the gasket may be the wearable device, or it may be the interface to the wearable device.
Diameter: as used herein, refers to a size of an opening or region. In some embodiments, the shape of the opening or region may be circular. In some implementations, the shape of the opening or region may be irregular and may define the major measurement of the opening or region. In some aspects, the diameter may be fixed and facilitate the interface between two objects of similar diameter. In some non-limiting exemplary embodiments, a diameter may comprise a cross section of an opening or region.
Supply Device: as used herein, refers to an interface between a wearable device and an external device that may supply a user with one or more predetermined items or substances such as oxygen, medical gases or liquids, as non-limiting examples. In some embodiments, the supply device may comprise a medical gas or liquid supply device.

The present disclosure describes systems and methods for reforming wearable devices. The system may comprise a wearable device. The wearable device may comprise a gasket. The wearable device may comprise a pliable region. The pliable region may comprise a predefined diameter to interface with a plurality of wearable devices. The pliable region may comprise the gasket. The wearable device may comprise a supply device. The wearable device may comprise an attachment mechanism.

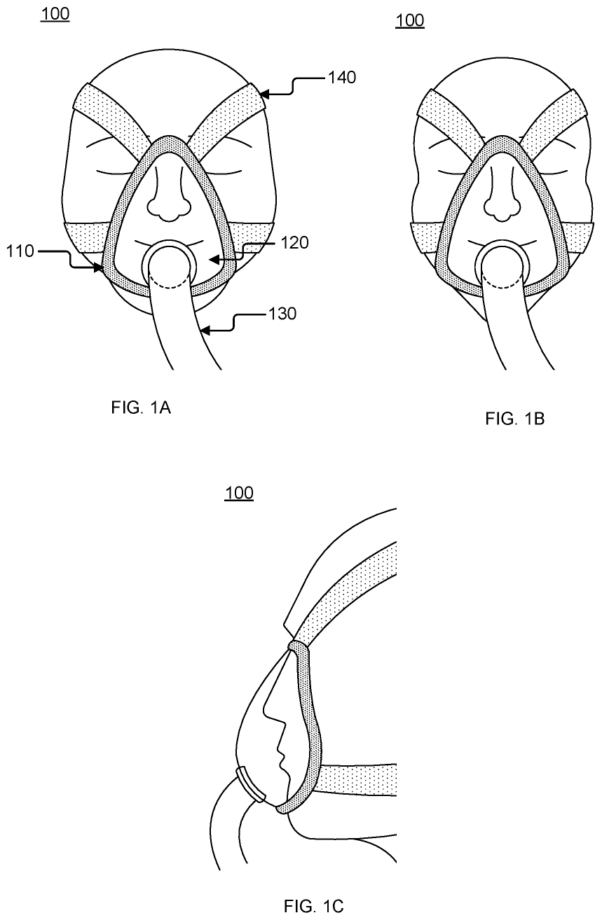

Referring now to FIGS. 1A-C, an exemplary wearable device 100 is illustrated. In some implementations, the wearable device 100 may comprise at least one pliable region 120. In some aspects, the wearable device 100 may comprise at least one supply device 130, wherein the supply device 130 may comprise an interface between the wearable device 100 and at least one external device configured to supply an amount of at least one predetermined item or substance to a user of the wearable device 100. In some embodiments, the wearable device 100 may comprise at least one attachment mechanism 140. In some aspects, the attachment mechanism 140 may be configured to at least temporarily secure the wearable device 100 to at least one portion of a user's body. In some embodiments, the attachment mechanism 140 and/or the pliable region 120 may securely or removably interface with at least one portion of the gasket 110. In some aspects, at least one portion of the wearable device 100 may be configured to become at least temporarily malleable when exposed to at least one external influence.

In some implementations, the wearable device 100 may be worn repeatedly for long-term purposes. In some aspects, the wearable device 100 may comprise a secure fit on the user's face. In some embodiments, the wearable device 100 may comprise a gasket 110. In some implementations, the gasket 110 may be configured to form a secure seal or fit with or upon at least one portion of an exterior or interior surface of the user's face or body. In some embodiments, the secure fit may become loose as a user's face changes.

For example, a long-term hospital patient may lose weight sufficient to alter the shape of their face because of their hospitalization. As another example, a child may require a face mask for breathing and the child's face may change as the child ages and matures. As another example, an elderly person may require face mask adjustments as the person continues to age and sagging changes the contours of the person's face. As another example, a patient with sleep apnea may require a larger face mask as they gain weight.

In some implementations, the wearable device 100 may be reformed to fit the new dimensions of a user's changed face. In some aspects, the wearable device 100 may be introduced to a reforming device. In some implementations, the reforming device may be configured to facilitate reshaping at least one portion of the wearable device 100. In some embodiments, the reforming device may be configured to exert at least one external influence on at least one portion of the wearable device 100 to at least temporarily convert the at least one portion of the wearable device 100 to a pliable or malleable state. By way of example and not limitation, the at least one external influence may comprise one or more of: an amount of heat, one or more chemicals, one or more wavelengths of light, one or more sound waves, an amount of radiation, at least one electrical current, or at least one applied force, as well as any combination thereof. In some embodiments, the wearable device 100 may be reshaped by the reforming device to match the new dimensions of a user's changed face. In some implementations, the reforming device may interface with an external device that assists in capturing the dimensions of the user's changed face and transmits those measurements to the reforming device. In some aspects, the wearable device 100 may receive thermal conditioning or undergo at least one phase change to soften the wearable device 100 sufficiently to reform the wearable device 100.

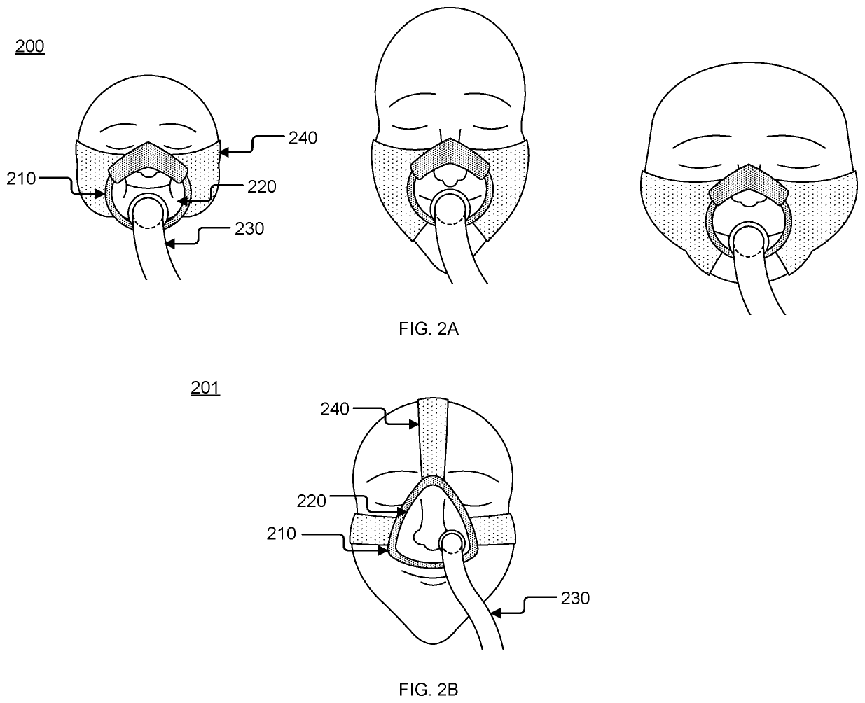

Referring now to FIGS. 2A-B, an exemplary wearable device 200 is illustrated. In some embodiments, the wearable device 200 may comprise a gasket 210. In some implementations, the wearable device 200 may comprise a pliable region 220. In some aspects, the wearable device 200 may comprise a supply device 230. In some embodiments, the wearable device 200 may comprise an attachment mechanism 240.

In some embodiments, the attachment mechanism 240 may be removable so as to be interchangeable. The interchangeable nature of the attachment mechanism 240 may allow the same wearable device 200 to be used for a plurality of users. In some aspects, the pliable region 220 may comprise a gasket 210. In some embodiments, the gasket 210 may be reformable.

As an illustrative example, an assisted breathing apparatus may be used in a hospital for patients that require a prolonged period of care. In some implementations, the attachment mechanism 240 may interface with a gasket 210 to allow a plurality of users to utilize the same supply device 230. In some aspects, the gasket 210 or pliable region 220 may comprise an adjustable portion, such as a padded nose bridge, that allows minor adjustments to improve the fit of the wearable device 200 for the user. In some embodiments, the user may be able to add or remove layers from the nose bridge to further adjust the nose bridge.

As another illustrative example, a fire department may own a plurality of face masks that interface with oxygen tanks. Each firefighter may have a personalized, interchangeable attachment mechanism 240. When a firefighter is finished with their shift, they may remove their attachment mechanism 240 from the face mask, thereby allowing the face mask to be reused as it interfaces with the next firefighter's attachment mechanism 240 to provide another personalized fit. In some aspects, the wearable device 200 may be used for structural cast development, progressive correctional therapy, diving masks, and pilot or flight masks, as a non-limiting list of examples.

In some implementations, the wearable device 200 may cover a portion of the user's face. In some aspects, the wearable device 200 may cover a user's mouth and nose. In some embodiments, the wearable device 201 may cover a user's nose. In some implementations, the wearable device 200, 201 may cover one or more of the user's airways in order to facilitate oxygen or other gaseous supply via the supply device.

In some aspects, the wearable device 200, 201 may supply liquid to the user. For example, the wearable device 200 may comprise a feeding or hydration device that provides sustenance for patients during prolonged recovery or surgical operations. The reformable nature of the wearable device 200 may allow for a customized fit for the device that secures the wearable device 200 firmly in place. In some embodiments, the reformable aspect of the wearable device 200 may increase the comfort of the user while wearing or using the wearable device 200.

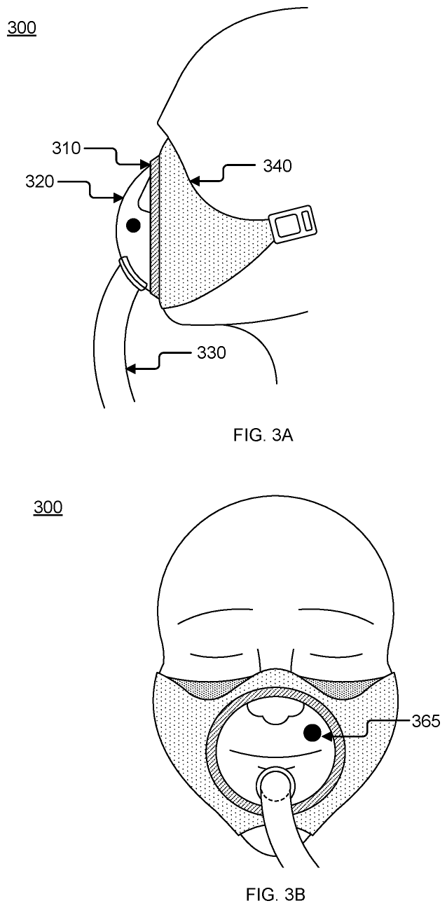

Referring now to FIGS. 3A-B, an exemplary wearable device 300 is illustrated. In some embodiments, the wearable device 300 may comprise a gasket 310. In some implementations, the wearable device 300 may comprise a pliable region 320. In some aspects, the wearable device 300 may comprise a supply device 330. In some embodiments, the wearable device 300 may comprise an attachment mechanism 340.

In some embodiments, the pliable region 320 may comprise a fixed diameter at the interface between the pliable region 320 and the gasket 310. In some implementations, the gasket 310 may interface between the attachment mechanism 340 and the pliable region 320. In some aspects, the pliable region 320 may comprise a plurality of fixed dimensions to ensure a secure interface with a supply device 330.

In some embodiments, the pliable region 320 may comprise a sensor 365. In some implementations, the sensor 365 may receive information from an external source. For example, the sensor 365 may receive information about a hospital patient's oxygen levels and may relay instructions to the wearable device 300 to dilate the opening of the supply device 330 to increase the amount of oxygen transferred to the patient.

In some aspects, the sensor 365 may transmit information about the user of the wearable device 300 to an external device. For example, the sensor 365 may monitor the user's CO and CO2 levels from their breath to regulate the amount of oxygen the user receives through the supply device 330.

As another example, the sensor 365 may detect respiration information, temperature, or moisture levels in the breath that may measure hydration levels in the body, and relay that information to a healthcare provider. In some embodiments, the sensor 365 may comprise biological or pathogenic sensors that may detect maladies such as pneumonia or possibly blood pressure. In some implementations, health information about the user may be collected by the sensor 365 and transmitted to an external source, such as a hospital or healthcare provider, as non-limiting examples. In some embodiments, the pliable region 320 may comprise a removable interchangeable manufactured component that may removably interface with a reformable gasket 310 or reformable attachment mechanism 340, or both. In some aspects, the gasket 310 or pliable region 320 may comprise an adjustable portion, such as a padded cheek liners, that allows minor adjustments to improve the fit of the wearable device 300 for the user. This adjustability may also be useful if the user's face changes over time, such as when a user has lost significant weight in their face. A user's facial contours may also change because of bloating, aging, and normal pediatric growth, as a non-limiting list of examples. In some embodiments, the user may be able to add or remove layers from the cheek liners to further adjust the padded cheek liners.

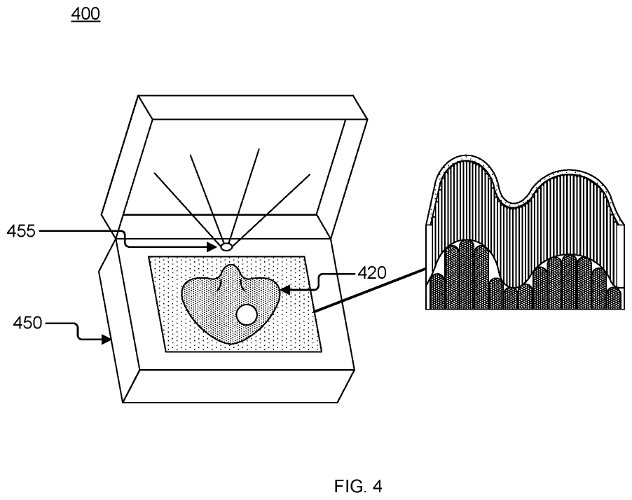

Referring now to FIG. 4, an exemplary wearable device 400 interfacing with a reforming device 450 is illustrated. In some aspects, the reforming device 450 may comprise a scanner 455. In some implementations, the wearable device 400 may interface with a reforming device 450.

In some aspects, the scanner 455 may record the existing dimensions of the user's face. In some embodiments, the pliable region 420 may be configured to become malleable when exposed to at least one external influence, such as, for example and not limitation, through heated, chemical, or other means. In some implementations, the reforming device 450 may assist in reforming the pliable region 420 for more complex curvature or a plurality of curves simultaneously.

As an illustrative example, the scanner 455 may measure the curvature of a user's face comprising the radius of the curvature of the bridge of the nose and the curvature of the face where the nose meets the region of the cheeks just beneath the eyes. The scanner 455 may transmit the measurements from this facial topology to the reforming device 450, which may then apply forming pressure to the malleable pliable region 420 sufficient to match the provided measurements.

In some aspects, the reforming device 450 may apply a variable amount of force on the pliable region 420 at a plurality of distances. In some embodiments, the reforming device may comprise a lid that may apply a compressive force to solidify a shaped pliable region 420. In some implementations, the lid may provide a protective cover to maintain the integrity of the shaped wearable device 400 as it stiffens.

As an illustrative example, the reforming device 450 may comprise a plurality of small pistons beneath a smooth, uniform surface. Each piston may extend to a different length to effectuate the dimension of curvatures received from the scanner 455. Separately actuated pistons may allow the reforming device 450 to form a plurality of contours and shapes, according to measurements or dimensions provided by the scanner or other non-limiting sources. The lid may close or remain closed to allow the wearable device's 400 shape to harden into the intended shape.

In some embodiments, the reforming device 450 may comprise a predefined shape. As an illustrative example, a face mask may become deformed over time and prolonged use as the material responds to cyclical use. The reforming device 450 may have a mold of the original face mask that allows the user to reform the face mask to its original dimensions. Alternatively, the reforming device 450 may reset the face mask to a generic size as part of a multi-step process to reform the face mask to a set of facial dimensions drastically different than the original.

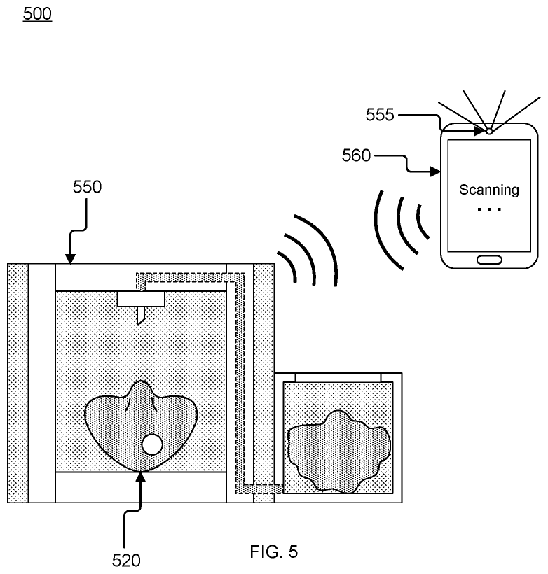

Referring now to FIG. 5, an exemplary wearable device 500 interfacing with a reforming device 550 is illustrated. In some embodiments, the wearable device 500 may comprise a pliable region 520. In some implementations, the wearable device 500 may interface with a reforming device 550. In some aspects, the reforming device 550 may interface with an external device 560.

In some embodiments, the external device 560 may comprise the scanner 555. In some implementations, the wearable device 500 may comprise an embedded scanner 555 that receives the dimensions of the user's face through positional awareness of contact points between the wearable device 500 and the user's face or scanning the user's face and creating a three-dimensional rendering by aggregating the collective measured distances from the scanner to the user's face, as non-limiting examples.

In some aspects, the scanner 555 may record the existing dimensions of the user's face. This scan may assist in shaping the wearable device 500 to at least partially match the dimensions of a user's face. In some embodiments, at least one portion of the wearable device 500 may be configured to become pliable or malleable when exposed to at least one external influence, such as, for example and not limitation, through heating or other means. In some implementations, the reforming device 550 may assist in reforming the wearable device 500 for more complex curvature or a plurality of curves simultaneously.

In some embodiments, the reforming device 550 may reduce the wearable device 500 to its composition materials. In some implementations, the reforming device 550 may reshape the wearable device 500 in a linear, gradient manner.

As an illustrative example, a specialized 3D printer may comprise an inlet tube connected to an adjacent heating chamber. The wearable device 500 may be placed within the heating chamber until the wearable device 500 has melted. The specialized 3D printer may reshape the wearable device 500 using the melted material. The 3D printer may print the dimensions of the reshaped wearable device 500 from measurements received from the scanner 555 on an external device 560.

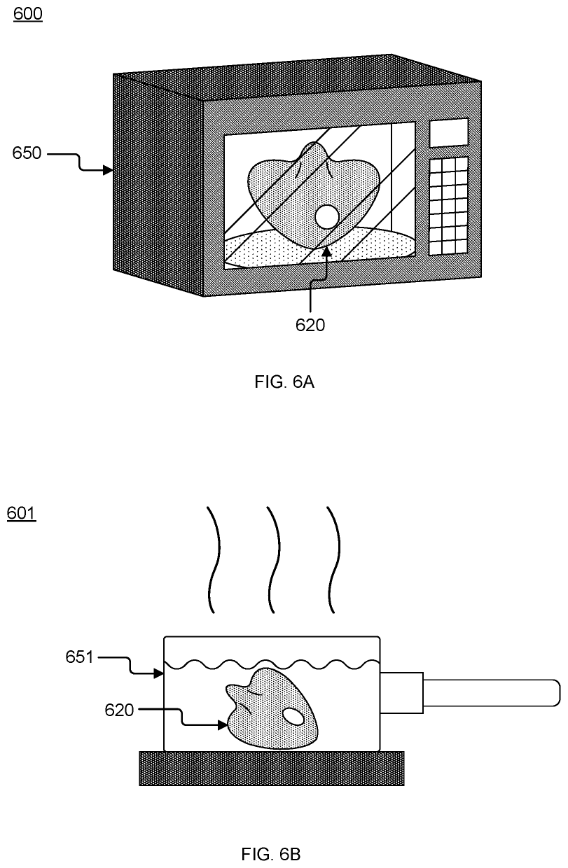

Referring now to FIGS. 6A-B, an exemplary wearable device 600 interfacing with a reforming device 650 is illustrated. In some embodiments, the wearable device 600 may comprise a pliable region 620. In some implementations, the wearable device 600 may interface with a reforming device 650.

In some embodiments, the reforming device 650, 651 may comprise at least one heating mechanism configured to apply an amount of heat to at least one portion of the wearable device 600, 601. In some implementations, at least one portion of the wearable device 600, 601 may at least partially comprise at least one material that is configured to become at least temporarily pliable or malleable when introduced or is otherwise exposed to high levels of heat or an amount of heat that comprises a temperature at or above a minimum threshold value sufficient to convert the at least one portion of the wearable device 600, 601 to a pliable or malleable state. By way of example and not limitation, at least one portion of the wearable device 600, 601 may at least partially comprise an amount of ethylene-vinyl acetate (“EVA”) or one or more similar thermoplastics or thermopolymers, as well as any combination thereof. In some non-limiting exemplary embodiments, the pliable region 620 of the wearable device 600, 601 may be configured to become at least temporarily pliable or malleable when exposed to high levels of heat.

As an illustrative example, a user may place the wearable device 601 in a pot of boiling water to make the wearable device 601 malleable. After being in the water for seven minutes, the wearable device 601 may be placed on a counter for a few minutes to cool enough to touch. The wearable device 601 may retain sufficient malleability to allow the user to press the wearable device 601 to their face to shape the wearable device 601 in accordance with the geometry of the user's face.

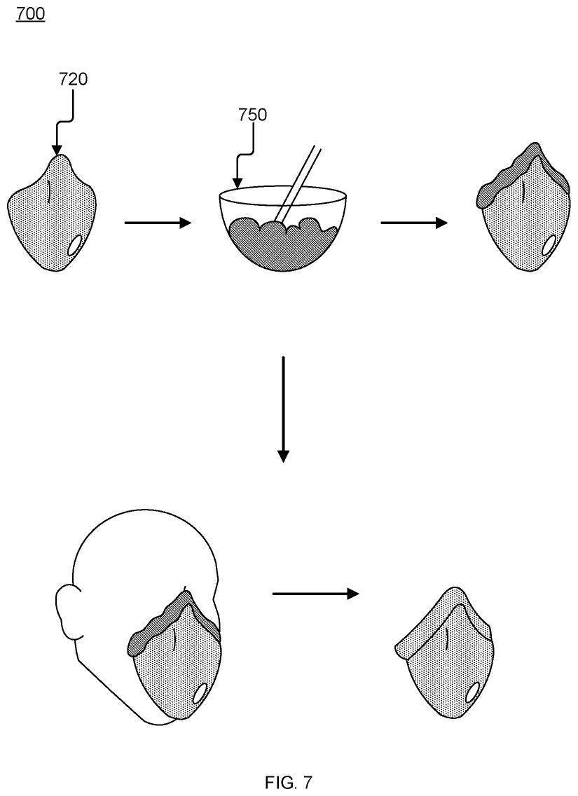

Referring now to FIG. 7, an exemplary wearable device 700 interfacing with a reforming device 750 is illustrated. In some embodiments, the wearable device 700 may comprise a pliable region 720. In some implementations, the wearable device 700 may interface with a reforming device 750.

In some embodiments, the wearable device 700 may comprise additional material. As an example, the user may add material to a face mask to improve the seal of the gasket. In some implementations, the user may add material to modify the shape, size, or fit of the wearable device 700, as non-limiting modifiable characteristics.

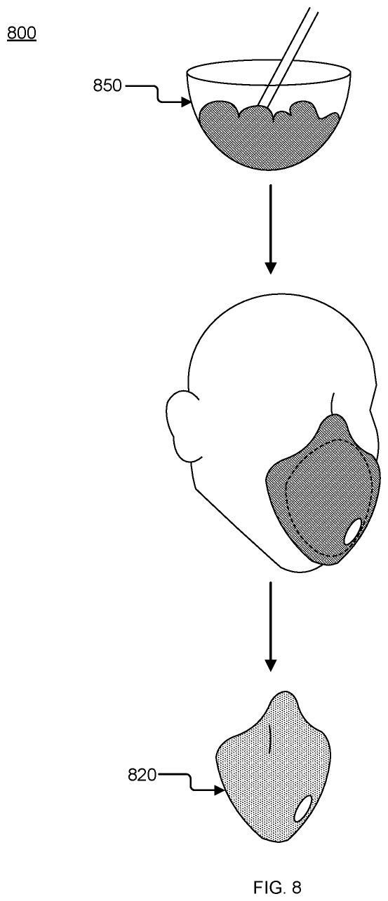

Referring now to FIG. 8, an exemplary wearable device 800 interfacing with a reforming device 850 is illustrated. In some embodiments, the wearable device 800 may comprise a pliable region 820. In some implementations, the wearable device 800 may interface with a reforming device 850.

In some embodiments, the wearable device 800 may comprise moldable, reformable material. In some implementations, the reformable material may, upon creation, be pressed upon the user's face to match the contours of the face. In some aspects, the reformable material may form a cast or mold that is used to form the wearable device 800. In some embodiments, the material may be reduced to pliable material to match the contours of a second face.

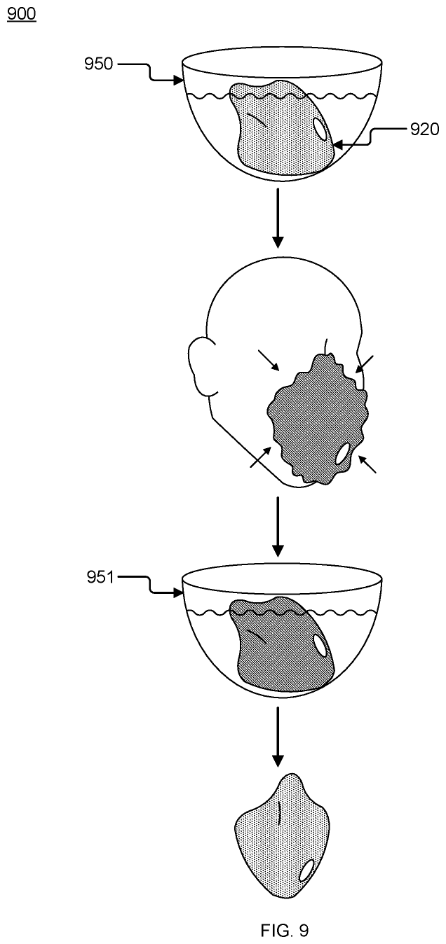

Referring now to FIG. 9, an exemplary wearable device 900 interfacing with a reforming device 950, 951 is illustrated. In some embodiments, the wearable device 900 may comprise a pliable region 920. In some implementations, the wearable device 900 may interface with a reforming device 950, 951. In some non-limiting exemplary embodiments, the reforming device 950, 951 may be configured to facilitate at least one interaction between at least one portion of the wearable device 900 and one or more chemicals, compounds, solutions, compositions, or similar substances, wherein the at least one interaction may cause at least one portion of the wearable device 900 to be at least temporarily converted to a pliable or malleable state. By way of example and not limitation, the reforming device 950, 951 may comprise at least one receptacle, such as a bowl, dish, or similar container, configured to at least temporarily receive an amount of one or more substances and at least one portion of the wearable device 900 such that the substance(s) may be able to interact with one or more portions of the wearable device 900 that may be exposed thereto.

In some embodiments, the wearable device 900 may become temporarily pliable or malleable when introduced or otherwise exposed to at least one substance that comprises at least one reagent solution. In some implementations, the reagent solution may chemically interact with at least one material of the wearable device 900. In some aspects, the reaction between the reagent solution and the material of the wearable device 900 may cause the wearable device 900 to become at least temporarily pliable or malleable. In some embodiments, the wearable device 900 may interact with the reagent solution for a predetermined period of time.

In some implementations, the user may press the wearable device 900 to their face to form an impression on the wearable device 900 that matches the area around the user's mouth. In some aspects, the reshaped wearable device 900 may be introduced to a hardening solution. In some embodiments, the hardening solution may allow the wearable device to solidify in its reshaped form.

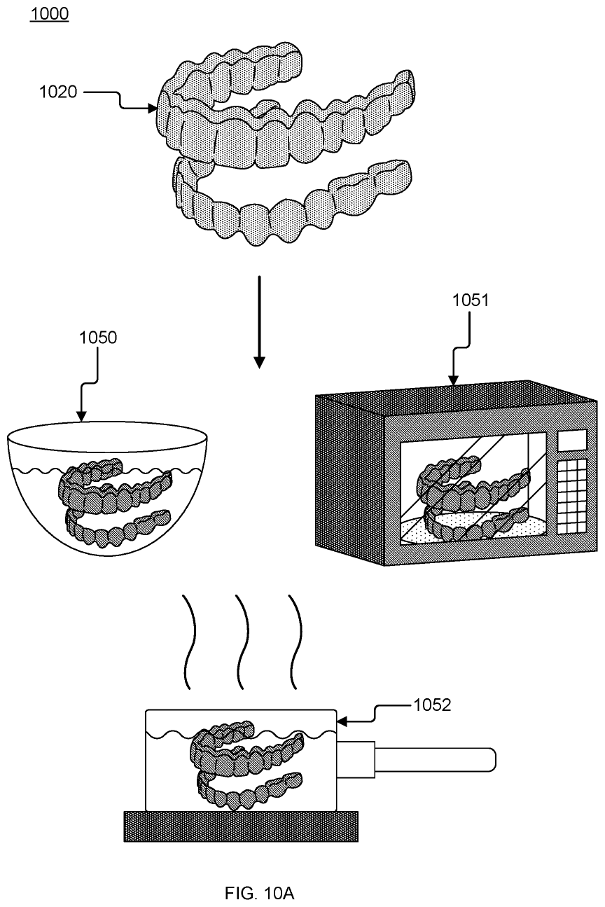

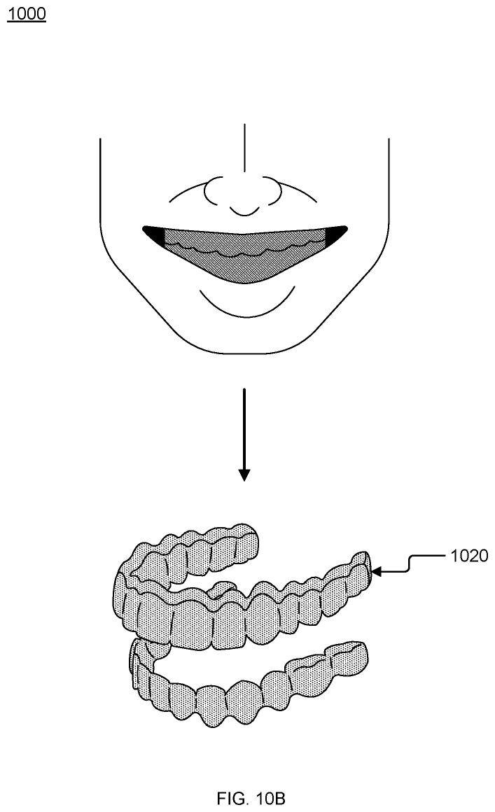

Referring now to FIGS. 10A-B, an exemplary wearable device 1000 interfacing with a reforming device 1050, 1051, 1052 is illustrated. In some embodiments, the wearable device 1000 may comprise a pliable region 1020. In some implementations, the wearable device 1000 may interface with a reforming device 1050, 1051, 1052.

In some embodiments, the wearable device 1000 may comprise a pliable region 1020. In some implementations, the wearable device 1000 may interface with a reforming device 1050, 1051, 1052.

In some embodiments, the reforming device 1050, 1051, 1052 may comprise at least one heating mechanism. In some implementations, at least one portion of the wearable device 1000 may at least partially comprise at least one material that becomes pliable or malleable when introduced to high levels of heat. In some embodiments, the required heat for malleability may be higher than the wearable device 1000 may be exposed to in normal use, thereby preventing the wearable device 1000 from becoming softened during normal use. In some implementations, the high heat may be just high enough to induce material softening. In some aspects, the portion of the wearable device 1000 that becomes pliable or malleable when exposed to high levels of heat may comprise the pliable region 1020.

As an illustrative example, a user may place thermopolymer-based retainer braces in a pot of boiling water to make the braces malleable. After being in the water for five minutes, the braces may be placed on a counter for a few minutes to become cool enough to touch. The braces may retain sufficient malleability to allow the user to place the braces within their mouth and use their teeth to reshape the braces. As the braces cool, the shape of the braces may be retained in accordance with the geometry of the user's teeth and mouth.

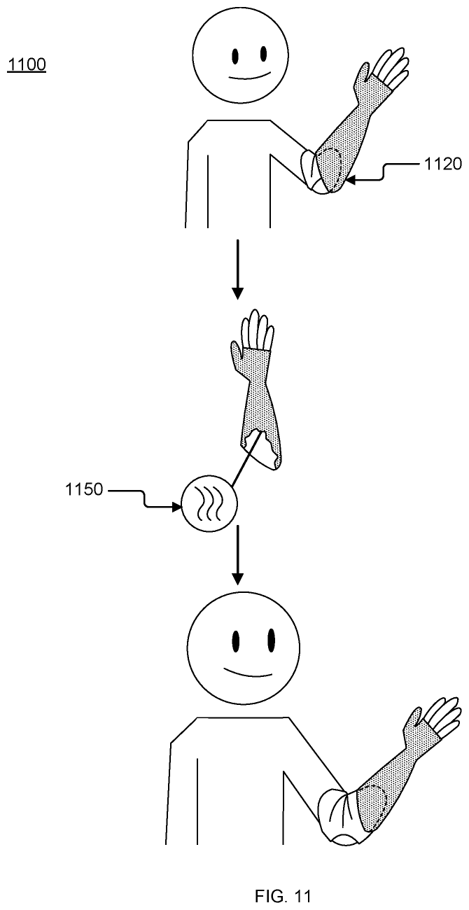

Referring now to FIG. 11, an exemplary wearable device 1100 interfacing with a reforming device 1150 is illustrated. In some embodiments, the wearable device 1100 may comprise a pliable region 1120.

In some embodiments, the reforming device 1150 may comprise at least one heating mechanism. In some implementations, at least one portion of the wearable device 1100 may comprise at least one material that becomes pliable or malleable when introduced to high levels of heat. In some embodiments, the reforming device 1150 may utilize a reforming process comprising an ultrasonic treatment, photonic treatment, electrical treatment such as nitinol or shape-memory wires, air-pressurized compressive systems, or a ferrofluidic treatment, as a list of non-limiting examples. In some aspects, the portion of the wearable device 1100 that becomes malleable may comprise the pliable region 1120.

As an illustrative example, a user may soften a portion of an under-sized prosthetic arm with a heat gun to make the prosthetic arm malleable. After several minutes, the prosthetic arm may be placed on a counter for a few minutes. When the contact surfaces are cool enough (or safe enough) to touch and the pliable core is still in its enhanced softened state, it can then be mated to the user's skin for achieving an optimum fit.

In some embodiments, extra material may be added to the prosthetic arm to increase the surface area. Increasing the surface area may be especially useful for a child who may be outgrowing a prosthetic arm and, by adding material, is able to correctly refit the existing prosthetic arm to a larger size for the growing child. After cooling, the prosthetic arm may retain sufficient malleability to allow the user to place the prosthetic arm on their arm to reshape the prosthetic arm. As the prosthetic arm cools, the reshaped form of the prosthetic arm may be retained in accordance with the geometry of the user's arm.

In some implementations, the wearable device 1100 may be used in progressive therapy. As an illustrative example, after a severe car accident, a recovery patient may have fractures that are too extreme to be absolved by a single cast or treatment. A cast might be created to hold a recovering limb in a predetermined position until the limb has healed sufficiently to increase the range of motion. The cast may then be reformed to maintain a second position that brings the injured limb closer to a position of complete recovery and full range of motion.

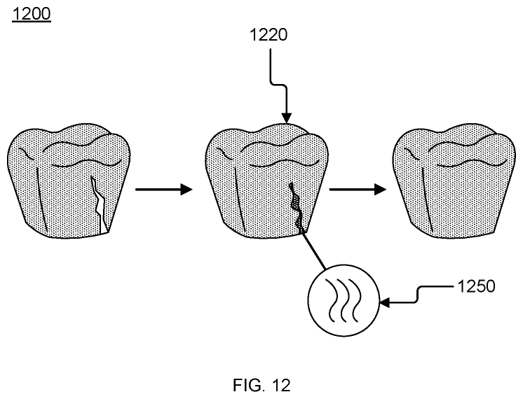

Referring now to FIG. 12, an exemplary wearable device 1200 is illustrated. In some embodiments, the wearable device 1200 may comprise a pliable region 1220. In some implementations, the wearable device 1200 may become malleable via a reforming device 1250.

In some embodiments, the reforming device 1250 may comprise at least one heating mechanism. In some implementations, at least one portion of the wearable device 1200 may comprise at least one material that becomes pliable or malleable when introduced to high levels of heat. In some aspects, the portion of the wearable device 1200 that becomes malleable when exposed to high levels of heat may comprise the pliable region 1220.

As an illustrative example, a user may place a cracked crown or other dental fixture in a pot of boiling water to make the crown malleable. After being in the water for several minutes, the crown may be placed under pressure to reform the crown. In some embodiments, extra material may be added to the crown to increase the surface area and fill cracks. After the crack has been remedied, the crown may be placed within the mouth and the user's teeth may reshape the crown to the teeth. As the crown cools, the shape of the crown may be retained in accordance with the geometry of the user's teeth and mouth.

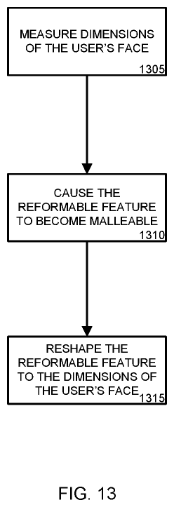

Referring now to FIG. 13, exemplary method steps for reforming wearable devices are illustrated. At 1305, one or more dimensions of one or more physical features of a user's face or other portion(s) of a user's body may be measured or otherwise obtained. In some embodiments, at least one scanner may be used to measure or otherwise obtain the dimensions of the user's face or body. In some implementations, an external device may comprise the scanner and the external device may be configured to transmit the measured dimensions to at least one reforming device. At 1310, at least one portion of the wearable device may be at least temporarily converted to a pliable or malleable state. In some aspects, the wearable device may become malleable as a result of applying an amount of heat. In some embodiments, the wearable device may become malleable as a result of at least one chemical or elemental interaction.

At 1315, the wearable device may be reshaped to at least partially match the dimensions of the features of the user's face or body. In some aspects, reshaping of the wearable device may be at least partially facilitated by at least one reforming device. In some non-limiting exemplary embodiments, a reforming device may impress the dimensions of the user's face or body on the wearable device. In some implementations, the dimensions of the user's face or body may be transmitted to the reforming device from a scanner or similar external device.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single product or packaged into multiple products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A wearable device comprising:
at least one pliable region; and
at least one gasket, wherein the at least one pliable region interfaces with the at least one gasket, wherein the at least one gasket is configured to form a seal with at least one portion of an exterior or interior surface of a body of at least one user, and wherein the at least one gasket interfaces with a diameter of the at least one pliable region.

2. The wearable device of claim 1, wherein the wearable device further comprises at least one supply device, wherein the at least one supply device comprises an interface between the wearable device and at least one external device.

3. The wearable of claim 2, wherein the at least one external device is configured to supply an amount of at least one predetermined substance to the at least one user.

4. The wearable device of claim 3, wherein the at least one predetermined substance at least partially comprises oxygen.

5. The wearable device of claim 1, wherein the wearable device further comprises at least one attachment mechanism, wherein the at least one attachment mechanism interfaces with the at least one gasket, and wherein the at least one attachment mechanism is configured to at least temporarily secure the wearable device to the at least one user.

6. The wearable device of claim 1, wherein the at least one pliable region is removable from the at least one gasket.

7. The wearable device of claim 1, wherein the at least one attachment mechanism is removable from the at least one gasket.

8. The wearable device of claim 1, wherein at least one portion of the wearable device is configured to become at least temporarily malleable when exposed to at least one external influence.

9. A system for reforming wearable devices comprising:
at least one wearable device, wherein the at least one wearable device comprises:
at least one pliable region; and
at least one gasket, wherein the at least one pliable region interfaces with the at least one gasket, wherein the at least one gasket is configured to form a seal with at least one portion of an exterior or interior surface of a body of at least one user, and wherein the at least one gasket interfaces with a diameter of the at least one pliable region; and
at least one reforming device, wherein the at least one reforming device is configured to facilitate reshaping of at least one portion of the at least one wearable device.

10. The system for reforming wearable devices of claim 9, wherein the at least one reforming device is configured to at least temporarily convert the at least one portion of the at least one wearable device to a malleable state.

11. The system for reforming wearable devices of claim 10, wherein the at least one reforming device comprises at least one heating mechanism.

12. The system for reforming wearable devices of claim 11, wherein the at least one portion of the at least one wearable device at least partially comprises at least one material that is configured to become at least temporarily malleable when exposed to an amount of heat that comprises a temperature at or above a minimum threshold value.

13. The system for reforming wearable devices of claim 12, wherein the at least one material comprises ethylene-vinyl acetate.

14. The system for reforming wearable devices of claim 10, wherein the at least one reforming device is configured to facilitate at least one interaction between the at least one portion of the at least one wearable device and one or more substances.

15. The system for reforming wearable devices of claim 14, wherein at least one of the one or more substances comprises at least one reagent solution.

16. A method for reforming wearable devices comprising:
obtaining at least one measurement of one or more dimensions of at least one physical feature of at least one user;
at least temporarily converting at least one portion of at least one wearable device to a malleable state; and
reshaping the at least one portion of the at least one wearable device to at least partially match the one or more dimensions of the at least one physical feature of the at least one user.

17. The method for reforming wearable devices of claim 16, wherein the at least one measurement is obtained via at least one scanner.

18. The method for reforming wearable devices of claim 17, wherein at least one external device comprises the at least one scanner, wherein the method further comprises:
transmitting the at least one measurement to at least one reforming device.

19. The method for reforming wearable devices of claim 16, wherein the reshaping of the at least one portion of the at least one wearable device is facilitated by at least one reforming device.

20. The method for reforming wearable devices of claim 19, wherein the at least one reforming device comprises at least one heating mechanism, wherein the method further comprises:
applying an amount of heat to the at least one portion of the at least one wearable device, wherein the amount of heat comprises a temperature at or above a minimum threshold value sufficient to at least temporarily convert the at least one portion of the at least one wearable device to the malleable state.
